(19) United States
(12) Patent Application Publication
(10) Pub. No.: US 2023/0071898 A1
(43) Pub. Date: Mar. 9, 2023

(54) APPARATUS AND METHOD FOR IRRADIATION

(71) Applicant: AquiSense Technologies, LLC, Erlanger, KY (US)

(72) Inventors: Jennifer Godwin Pagan, Charlotte, NC (US); Steven Franklin Pugh, Charlotte, NC (US); Richard Mark Simons, London (GB)

(21) Appl. No.: 17/466,251

(22) Filed: Sep. 3, 2021

(51) Int. Cl. A61L 2/10; G01N 21/64; A61L 9/20

(52) U.S. Cl. CPC A61L 2/10; G01N 21/64; A61L 9/20; A61L 2202/11; A61L 2209/12; A61L 2202/121; A61L 2202/14; A61L 2209/111; A61L 2202/122

(57) ABSTRACT

An apparatus and method for irradiating a fluid containing a material to be irradiated, comprising at least one irradiation chamber having at least one inlet port, one or more UV radiation sources inside the irradiation chamber(s) optically coupled to the fluid in the irradiation chamber(s) via at least one UV-transparent window in contact with the fluid; one or more seals or gaskets disposed adjacent to the radiation sources to protect them from the fluid; and at least one heat exchange mechanism inside the irradiation chamber(s) thermally coupled to the radiation sources and to the fluid. The UV radiation sources and the at least one heat exchange mechanism are at least partially submerged in the fluid in the irradiation chamber.

### FIELD OF THE INVENTION

The present invention relates generally to apparatus and methods for disinfection of fluids by irradiation. More specifically, the invention relates to an apparatus and methods for disinfection of fluids containing a material to be irradiated using one or more UV radiation sources.

### BACKGROUND OF THE INVENTION

The use of ultraviolet (UV) radiation for the purpose of disinfection of a fluid, including liquids and gases, is well known. The process of using ultraviolet radiation to inactivate microbial contaminants in fluids is referred to as Ultraviolet Germicidal Irradiation (UVGI). Ultraviolet radiation has also been used for oxidizing organic and inorganic materials in a fluid, termed Advance Oxidation Process (AOP), and many commercial AOP systems are in use today. Systems employing UVGI and AOP methods rely on the ability to transmit UV radiation into the fluid in a predictable manner.

Both AOP and UVGI require a UV source. For practical purposes, the output irradiance of the UV source should be maintained and decay in a predictable manner over the usage lifetime of the UV source. This enables predictions about the replacement cycle of the UV source as well as the overall performance of the system. Some NSF and EPA regulations require UV disinfection systems to be tested with the UV source operating at predicted End of Lamp Life (EOLL) optical output power. In order to adhere to the UV disinfection system performance specifications for a predicted time period, the UV source should decay in a predictable manner. There are also commercial benefits to having longer EOLL, which leads to longer system lifetimes and/or UV source replacement intervals.

There are many types of UV sources. Historically, low pressure mercury vapor lamps, medium pressure mercury vapor lamps, and amalgam lamps have been used as UV sources for disinfection applications. Other UV sources include deuterium lamps, light emitting diodes (LEDs), lasers, micro plasma sources and solid-state field effect phosphor devices. Micro plasma lamps operate on the same principle as the large gas discharge lamps but have a planar electrode generating small localized pockets of UV emission. Solid state sources such as LEDs create light in a semiconductor material through charge recombination in an active layer where carrier injection is applied to an anode and cathode of the semiconductor heterostructure. All of these UV sources have different optimal operating temperatures where the UV output flux and/or the lifetime is maximized. Most gas discharge lamps are difficult to operate in very cold ambient conditions because of reduced mercury vapor pressure. Conversely, solid state sources have maximized outputs at ambient temperatures lower than those of mercury vapor lamp For example, the output power of a low-pressure mercury lamp may peak at an ambient temperature of 40 degrees Celsius while the optical output power of a 265 nm LED displays a linear relationship with ambient temperature. The slope of the LED curve may vary by the device design, but the trend remains the same with larger optical output powers seen at lower ambient temperatures.

All UV sources produce waste heat, the wall plug efficiency of UV LEDs for instance is currently lower than 10%. This means that more than 90% of the input power to the device is not converted to UV photons but to waste heat. Even more mature technologies, such as mercury vapor lamps, are less than 40% efficient, illustrating that waste heat management remains a concern throughout the technology development cycle. When packaging a light source for use, provisions must be made to handle the waste heat. This can be accomplished through passive convection in air. However, as the power and number of LEDs used inside a lamp increase, the heat load could be too large for reasonable operation of the LEDs. Alternately, the ambient temperature could be too hot for the passive convection to be effective in reducing the LED temperature.

Many LED manufacturers specify a maximum junction temperature which should not be exceeded during operation. The LED junction temperature is the temperature of the active layer sandwiched between the n-type and p-type semiconductor layers of the LED. Exceeding a maximum rated junction temperature may result in a degradation in the lifetime or other characteristics of the LED. In a simplified model, an LED can be represented as a series of thermal resistances. For example, a UV LED package may be a surface mount device (SMD) mounted onto a circuit board, which is itself mounted onto a heatsink or other cooling device. The heatsink may be any heat exchanger or method of cooling, such as a passive heatsink, Peltier device, active airflow, heat pipe, etc. The LED may be mounted on a variety of electrically and thermally conductive circuit boards, such as a printed circuit board (PCB), a metal core printed circuit board (MCPCB), or a chip on board (COB). Every point of connection from the junction of the LED itself to the ambient environment has a temperature gradient associated with it. These include the junction temperature of the LED, the temperature between the LED package at the circuit board, the temperature between the circuit board and the heatsink, and the ambient temperature. At each point of connection, one can model a thermal resistance (° C./W), such that R_{JS} is the thermal resistance of the surface mount LED package, R_{SB} is the thermal resistance of the circuit board, and R_{BA} is the thermal resistance of the heatsink or cooling method. The LED junction temperature can be modeled as the ambient temperature added to the sum of each of the thermal resistances multiplied by the power lost to heat in the device. This relationship is shown in Equation 1.

T_{J(LED)}=T_{Ambient}+Σᵢ(Rᵢ×P_{Heat}) Equation 1

LEDs are unique among most TV sources in that heat is removed through the side of the chip which is electrically connected to power, versus the side which is responsible for most of the UV emission. This is in contrast to a mercury vapor lamp, which has a thermal discharge predominantly in the same direction as light emission through a quartz sleeve, which also functions to contain the plasma as the arc discharge tube. LEDs do not require a quartz window as they emit light directly from the active layer of the semiconductor, and the light transmits through the epitaxial and substrate layers to exit to the ambient. However. LEDs can be sensitive to electro-static discharge, moisture, and ambient gases like oxygen or nitrogen which can degrade the performance of the LED electrical contacts and the semiconductor. For this reason, a quartz window is often placed on the SMD package of a LED. In UVGI systems where the LED will be protected from the fluid via a window, the window on the SMD becomes superfluous if the above environmental impacts can be mitigated. A single window over a board containing one or more LEDs can be used as the optical window for a fluid disinfection system if the LEDs are sealed between the board and the window such that the window can serve as a portion of the pressure vessel for the disinfection system and to segregate the LEDs from the fluid. Potting compounds like epoxies or silicones can be used between the board and the window to accomplish this; similar sealing of the space around the LED may be achieved with suitable use of gaskets or other mechanical seals. The potting may be undertaken in a low relative humidity environment or even purged with dry air or an inert gas to ensure any voids between the LED and window do not have undesirable moisture or gases inside. This would also increase the output power of the LED since it would pass light through one quartz window versus two. An additional benefit to this type of single window lamp package is that the LED imparts little heating to the window, in contrast to mercury vapor sources which transmit a large amount of heat to the window. Lower window temperatures have been correlated to less fouling of the window. Window fouling lowers the overall UV transmittance of the window, which in turn lowers the performance of UVGI and AOP systems. Thus, a robust product design utilizing a UV source will account for the temperature of the UV source during operation by consideration of heat transfer. By such methods the lifetime and output power of the UV source may be better controlled. In addition, methods of assembling the UV source into secondary packaging can be used to enhance the output power, lifetime, and effective performance of the UV source.

While the UV source is an important component in a UVGI system, it is only one component in the overall system efficiency. The system efficiency can be expressed as the product of the reactor efficiency and the UV source efficiency. It is good practice in the design of a UVGI system to maximize the exposure time, often termed the “residence time”, of the fluid to the UV irradiance thereby maximizing the dose seen by the fluid. The reactor efficiency is a combination of the residence time efficiency and the optical efficiency. The optical efficiency of the reactor is a measure of how effectively the reactor uses photons from the UV source to increase the probability that a microbial contaminant in the fluid will absorb a photon. One method of increasing this probability is to use reflective materials in the reactor such that photons from the UV source may be reflected if they are not absorbed during their initial pass inside the reactor. If there are few absorbers in the fluid and the reflectivity of the material in the reactor is high, the photons may be reflected multiple times inside the reactor. The use of reflective materials, and resulting multiple passes through the reaction chamber, have additional benefits of improving the uniformity of irradiation of the fluid; this may be framed, similarly, as the probability that a microbial contaminant within the reaction chamber may absorb a photon, since increased uniformity of irradiation (fluence rate) throughout the reaction chamber reduces the spatial and temporal variation in photon flux that a target microbe might experience.

Microbes are prolific in the environment and can multiply and even form biofilms, both of which can present a health hazard for humans or interrupt intended processes. Products such as coffee makers, water servers, and chiller tanks use reservoirs to store water for human consumption or other processes, such as in manufacturing. Even when loaded with potable or filtered water, reservoirs may contain sufficient nutrients for microbial proliferation and biofilm growth; further, contamination may be present in the tank prior to the loading or potable water, or may be introduced at a later date from ambient sources or otherwise. Biocides are often used in process waters to inhibit biofilms and microbial contaminants from propagating in storage tanks and distribution lines. Many public drinking water distribution systems use chlorination to chemically disinfect water and provide a residual disinfectant. However, biocides may lose their effectiveness over time and need to be replenished, leaving water reservoirs vulnerable to microbial growth. Even with continual application and monitoring, sterility is rarely accomplished and cannot be expected beyond a few specialist cases such as laboratories, surgical equipment, and the production of pharmaceuticals: microbial contamination is, therefore, an ever-present concern across the built and natural environments.

Compact ultraviolet sources which provide a germicidal effect to surfaces, gases, gels, liquids, and other fluids or solids are increasingly commercially available at a range of optical output powers, from sub milli-Watt to several Watts; arrays of such sources can therefore be formed from milli-Watt to kilo-Watt output and beyond. The availability of these sources, including light emitting diodes (LEDs), plasma lamps, and solid-state emitters, has led to an increase in the use of such devices for pathogen inactivation in a variety of products. LED based sources are particularly useful due to their low DC voltage requirements, instant on/off operation with power application, and compact size. While the use of ultraviolet sources for storage tank disinfection and biofilm inhibition is desirable, there are a number of challenges to implementation.

One challenge is packaging the ultraviolet source such that it is protected from fluid in the reservoir it will be used to disinfect. Gaskets and seals can be employed to provide protection from the fluid while maintaining a UV transparent area to allow the UV radiation to expose the reservoir fluid and/or surfaces. These ultraviolet sources can be packaged to various levels of ingress protection to separate electrical and electronic components of the UV source from the potentially damaging environment of the tank (water and other liquids, humidity, vapors, gases, dust and debris, etc.).

Another challenge is that many sources have optimal operating temperature ranges. Ultraviolet LEDs (UVLEDs), for instance, have maximized optical outputs at lower temperatures, and operating the LED at higher temperatures causes a larger drop in optical output power over a given period. This is an accepted attribute of UVLEDs, with most LED manufacturers specifying a maximum LED solder or junction temperature that should not be exceeded for their devices. While vapor discharge lamps emit heat through the glass envelop surrounding the arc of the lamp, semiconductor UV sources such as UVLEDs emit heat through the portion of the device where the anode and cathode electrical connection is made. Typically, this is through an electrical connection made to a surface mount device package or directly by having the LED mounted on a circuit board. This means that while the UV photons are emitted from all around the LED, the majority of the photons come from a 180 degree or smaller angle of emission, since the electrical connection to the LED is typically opaque. The implication is that the majority of the heat is conducted through the electrical connection of the LED while the UV emission is majority through the opposing direction. Thermal management is thus typically performed through the non-emitting side of a LED lamp where it has the largest impact on the junction temperature of the LED. Thermal management is a key criterion of any beneficial design.

Yet another challenge to using UV radiation to effectively disinfect reservoirs is the effective distribution of UV radiation through the water volume. Shadowed regions and ‘dark spots’, where relatively few photons from the UV source penetrate, may not achieve sufficient disinfection even after long exposure periods, and can be significantly limiting to an overall system efficacy. The negative impacts of ‘dark spots’ in UVGI apparatus, whereby the apparatus has limited effect on a portion of the target media, and the impacts on efficacy and efficiency in germicidal applications has been reported in the literature. Since fluid in a tank may be stagnant for long periods of time, microbes may grow in shadowed regions as the fluid is not mixed. The mixing of fluid in reservoirs that have shadowing or that have a non-uniform UV radiation inside is one means to ensure the entire volume is disinfected. Although this may not be sufficient as a means for biofilm control, it may delay formation of biofilms through minimizing the microbial load in the fluid volume. Outside of theoretical designs, inhomogeneity of UVGI through a target volume is an unavoidable attribute of such systems and negatively impacts efficacy: the appropriate and effective mixing of such fluids may circulate material to be irradiated from ‘dark spots’ into regions of higher exposure, and so is a means to increase treatment efficacy.

One means for preferentially altering the delivery and distribution of UVGI within a tank system is by the deliberate positioning of the UV source such that the radiation transmitted through the window is preferentially directed towards a specific target region, or to reduce non-uniformity across the whole. Displacement of the UV source from the reaction chamber wall may enhance this ability, and be achieved by use of a stem support; such projection may further enhance the thermal transfer efficiency by affecting the ability of fluid to flow around the thermal transfer surface. Optimum positioning of the UV source within the 3-dimensional fluid volume will vary depending on the disinfection objective (e.g. biofilm inhibition across chamber surfaces, bulk disinfection of the fluid within the tank, targeted treatment of a portion of the fluid), the irradiation chamber geometry, reflectivity of the interior surfaces, thermal management considerations, and other operating considerations.

U. S. Patent Application Publication 2014/0161664 A1 and U.S. Pat. No. 10,500,295, both incorporated herein by reference, disclose various apparatus, materials, and methods useful for disinfection of fluids by irradiation. However, there is a continuing need for an irradiation apparatus and method useful for treating or maintaining microbial quality in a variety of fluid housings or flow cells, particularly potable water tanks, that provides good efficiency and thermal management while maintaining a compact footprint. The present invention addresses the integration needs for more generalized irradiation apparatus, such as in the treatment of potable water storage tanks where native fluid flow may be slow, intermittent, or ineffective at mixing the whole fluid volume.

### SUMMARY OF THE INVENTION

In one embodiment, the invention relates to an irradiation apparatus comprising at least one irradiation chamber for a fluid containing a material to be irradiated, said chamber having at least one inlet port for fluid flow into the chamber; one or more UV radiation sources inside the at least one irradiation chamber optically coupled to the fluid in the at least one irradiation chamber via at least one UV-transparent window in contact with the fluid in the irradiation chamber; one or more seals or gaskets disposed adjacent to the one or more radiation sources to protect the one or more radiation sources from the fluid in the irradiation chamber; and at least one heat exchange mechanism inside the at least one irradiation chamber thermally coupled to the one or more radiation sources and to the fluid in the at least one irradiation chamber; wherein the one or more UV radiation sources and the at least one heat exchange mechanism are at least partially submerged in the fluid in the irradiation chamber.

In another embodiment, the invention relates to a method for irradiating a fluid containing a material to be irradiated disposed in an irradiation chamber, the irradiation method comprising (1) providing an irradiation apparatus comprising at least one irradiation chamber for a fluid containing a material to be irradiated, said chamber having at least one inlet port for fluid flow into the chamber; one or more UV radiation sources inside the at least one irradiation chamber optically coupled to the fluid in the at least one irradiation chamber via at least one UV-transparent window in contact with the fluid in the irradiation chamber; one or more seals or gaskets disposed adjacent to the one or more radiation sources to protect the one or more radiation sources from the fluid in the irradiation chamber; and at least one heat exchange mechanism inside the at least one irradiation chamber thermally coupled to the one or more radiation sources and to the fluid in the at least one irradiation chamber; wherein the one or more UV radiation sources and the at least one heat exchange mechanism are at least partially submerged in the fluid in the irradiation chamber, and (2) irradiating a fluid containing a material to be irradiated using said irradiating apparatus.

### BRIEF DESCRIPTION OF THE DRAWINGS

The invention is illustrated and described herein with reference to the various drawings, in which like reference numbers are used to denote like apparatus components, as appropriate, and in which:

FIG. 1 is a planar side view illustrating one exemplary embodiment of the irradiation apparatus of the invention:

FIG. 2 is a section view of the apparatus of FIG. 1 taken along line 2-2;

FIG. 3 is a section view of the apparatus of FIG. 1 taken along line 2-2 and illustrating convective cooling currents induced in the fluid in the irradiation apparatus;

FIG. 4 is an enlarged view of a portion of the irradiation apparatus shown in FIG. 3.

### DETAILED DESCRIPTION OF THE INVENTION

The present invention provides a UV irradiation apparatus, disinfection system and method comprising at least one irradiation chamber for fluid containing a material to be irradiated, and one or more UV radiation sources inside the irradiation chamber optically coupled to the fluid in the irradiation chamber via at least one UV-transparent window in contact with the fluid. One or more seals or gaskets are disposed adjacent to the radiation sources to protect them from the fluid in the irradiation chamber. At least one heat exchange mechanism inside the irradiation chamber is thermally coupled to the radiation sources and to the fluid in the irradiation chamber. The UV radiation sources and the heat exchange mechanism are at least partially submerged in the fluid in the irradiation chamber. Optionally, the heat exchange mechanism may not be of suitable material for contact with the fluid for human consumption or for fluid used in a medical process. In this case the portions of the heat exchange mechanism exposed to the fluid may be coated with a material approved for drinking water, food contact or medical material compatibility.

The UN irradiation apparatus, disinfection system and method are designed such that at least a portion of the radiation from the one or more radiation sources is transmitted to surfaces of the at least one irradiation chamber to provides a disinfection effect to inhibit the propagation of microbiological contamination thereon. Microbial attachment to surfaces of the irradiation apparatus, hereinafter referred to as “biofilm” formation, may increase risk to health due to possible transfer of such contaminants to a fluid flowing across such surfaces, or spontaneous transfer may be possible. The inhibition of biofilm within the disinfection system is desirable since the process of UV irradiation does not impart a residual biocide to the fluid treated. In one embodiment, a small portion of the radiation emitted by the UV source may be redirected to irradiate surfaces of the treatment apparatus and system. Since the fluid-contact surfaces of the reactor are static, the irradiation period of any segment is equal to the total period for which the UV source is emitting. Thus, far lower irradiances are required to achieve biofilm inhibition than would be necessary for transient irradiation, such as for a fluid passing through a reactor chamber. By requiring low irradiance and relatively low UV power, a small fraction of the power emitted by the source can be scavenged for biofilm inhibition without significantly impacting the fluid disinfection performance of the reactor. Thus, a portion of the radiation from the one or more radiation sources can be transmitted to surfaces of the one or more irradiation chambers to inhibit biofilm formation thereon.

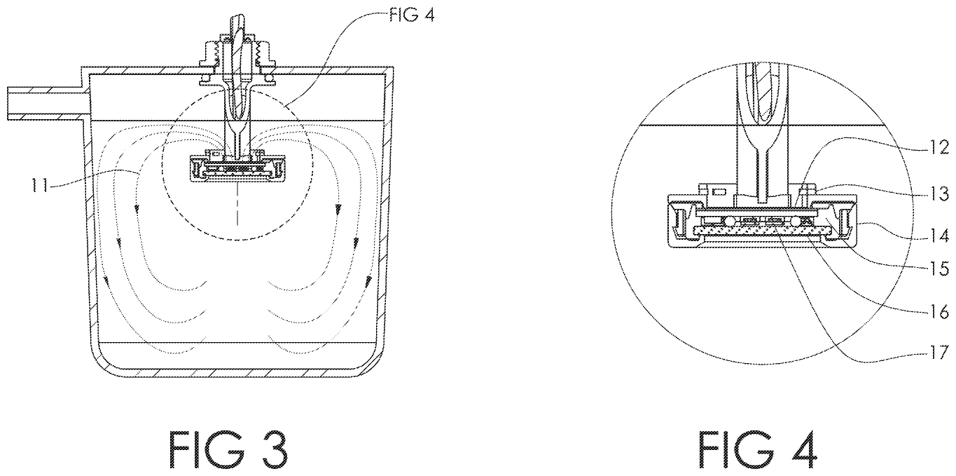

The above considerations motivate the design of a system for mounting the UV radiation source such that both the UV-transparent window and the at least one thermal exchange mechanism are wetted by a liquid, which may or may not be the intended irradiation target fluid. In the specific case of disinfection of a water tank, the UV source and protective housing are at least partially submerged in the water, providing both good optical and thermal coupling of the UV source to the target fluid. However, the location of the UV source within a water volume or the structure of the irradiation chamber itself may result in a portion of the water volume not receiving sufficient UV radiation exposure for disinfection. Beneficially, the heat generated by the UV source, and any materials thermally connected to the UV source, may induce convective currents within the fluid volume in an otherwise stagnant tank. These currents may circulate the fluid from the occluded regions into those of greater UV exposure, as such producing a more uniform and effective disinfection effect. Further, the components of the UV source intended for protection of the source from the environment may be designed in such a way to enhance convective cooling and mixing of the fluid volume. Structures may be added to the housing of the UV source to preferentially direct or increase the velocity of the convection currents. This is similar in concept to how thermal chimneys work in buildings with air, except in this case the currents are being induced in the water. In one embodiment of the invention, this convection effect is shown in a model of flow velocity in a static tank in FIG. 3.

The UV radiation source (or a plurality of UV radiation sources) may comprise one or more UV-C radiation sources, or a combination thereof. The UV radiation source (or plurality of UV radiation sources) is typically coupled to a support structure inside the at least one irradiation chamber. The support structure holds the UV radiation source(s) such that they selectively direct UV radiation into the interior of an irradiation chamber in which a material to be irradiated is disposed. Peak wavelengths may be (dynamically) selected and/or adjusted, and a plurality of wavelengths may be utilized such that the action spectrum of a given organism can be targeted, thus improving disinfection efficiency. For example, one or more wavelengths of the one more UV radiation sources may be selected based on an identification of a contaminant in the material to be irradiated. The one or more UV radiation sources may deliver one or more wavelengths, or a combination of wavelengths, to the material to be irradiated. The wavelengths may induce fluorescence in the material to be irradiated thereby allowing for the identification of the contaminant in the material to be irradiated. Optionally, the material to be irradiated may be disposed adjacent to an n-type single crystalline semiconductor to generate hydrogen peroxide at the semiconductor surface through bandgap electric photo-excitation for disinfection.

Heat in the irradiation apparatus is managed, and optionally recuperated, using a heat exchange mechanism, such as a one or more of a printed circuit board, a metal core printed circuit board, a thermoelectric cooling device, a vapor chamber, a heatsink, a heat dissipation structure, a thermal transfer material, and a material thermally coupled to a fluid, in contact to the UV radiation source(s). The irradiation apparatus may be made moisture resistant using a moisture seal coupled to and/or disposed within the support structure. The irradiation assembly can include a monitoring/detection mechanism and control circuitry for dynamically controlling the delivery of UV radiation to the material to be irradiated based on flow rate, water quality, user input, sensor readings, or other operating conditions. Finally, associated performance data may be stored in an onboard or external data storage unit and used to feedback signal to monitoring circuitry to deliver system status. The system status could be indicated by a current or voltage signal linked to a visible or audible alarm.

In various embodiments of the invention, a modular semiconductor UV LED mounting configuration may be provided including a UV radiation source package containing a single LED or multiple LED “dice” arranged in a matrix or array. The LED dice can be selected to provide multiple wavelengths in both the UV and visible radiation spectrum from about 200 nm to about 800 nm. In one exemplary embodiment, the matrix or array includes LED dice emitting wavelengths in the range of about 200-320 nm in order to saturate the absorption mechanism of nucleocapsids (with peak emission centered at around 280 nm), and at the same time to target the peak absorption of nucleic acid with its peak emission wavelength spanning about 250-280 nm. In another exemplary embodiment, with the intention of mimicking the optical output spectrum of low or medium pressure Hg-based UV lamps used to target various bacteria and viruses, the matrix or array of LED dice utilizes multiple wavelengths, including at least one of about 240-260 nm, about 260-344 nm, about 350-380 nm, about 400-450 nm, or about 500-600 nm. A further exemplary embodiment is a matrix or array of LED dice emitting germicidal wavelengths ranging from about 250 nm to 300 nm in conjunction with LED dice emitting wavelengths in the range of about 350 nm to 400 nm to enable photocatalytic oxidation of pathogens or pollutants in water in proximity of crystalline films of n-type semiconductors, such as TiO₂, NiO, or SnO₂. A still further exemplary embodiment is a modular mounting configuration containing multiple LED dice emitting about 250-320 nm and about 320-400 nm wavelengths arranged in a matrix or array to enable the fluorescence spectra of NADH, and tryptophan, of particles with biological origin. In another exemplary embodiment, a commercially available SETi UV Clean™ LED package is used. Individual LED dice or a single die bonded to a thermally conductive metal core circuit board (MCPCB), such as those available from The Bergquist Company™, may also be used.

A packaged UV LED, or a matrix or array of multiple UV LEDs, may be attached to the heatsink. Multiple UV wavelengths can be used to optimize the effect on specific microorganisms. Backside heat extraction may be aided by thermoelectric cooling (TEC) and/or a vapor chamber. Additionally, the UV LED package may be topside cooled by conduction through a highly thermally conductive over-layer, such as silicone polymer impregnated with diamond nanoparticles, which may have a single crystalline structure.

Components for the electrical and/or electronic control of the TV radiation source may optionally be included within the sealed unit as previously described, such that they may act upon the UV radiation source whilst maintaining protection from the external environment through such hermiticity, the use of desiccants, or a combination thereof as previously described. Further, the co-location of these components onto the MCPCB, or otherwise, and subsequent thermal union to the heat exchange mechanism may be used to extract heat generated by, for example, power conversion components. Additionally, these electrical and/or electronic components may include sensors by which the operating conditions and status of the UV radiation source may be determined, including but not limited to a photodiode, thermocouple, thermistor, acoustic sensor, hall probe, current probe, etc.

The radiation emitter module may be a user replaceable unit, optionally including attached electronics and desiccating materials in order to combat moisture and humidity. Attached electronics can include an individual identification number and telemetry tracking, as well as an interconnect for easy disconnect from a larger system.

The UV radiation may be transmitted from the LED dice through a transmissive window, polymer, air, and/or aperture into the irradiation chamber. The transmissive window has a transmission spectrum appropriate for the choice of LEDs used, for example the UV-C range.

Fused silica, fused quartz, or similar glasses, are commonly used for this purpose, as are UV-stable silicones (e.g. DOW Silastic, LEDiL VIOLET). These window materials therefore constitute part of the optical coupling system and their efficiency at transferring light from the source to the target medium will affect the overall system efficacy. The Fresnel equations are well understood in their description of the transmission efficiency across refractive index boundaries. In the case where a UV source is positioned such that the window is ‘dry’, i.e. not in contact with the water volume of a storage tank, the UV radiation must cross three large refractive index boundaries (air-quartz-air-water), and subsequently undergoes three substantial loses in transmitted power (due to the reflected portion). For such an air-quartz-air-water system, up to 9.8% of the UV radiation emitted by the source would not reach the water target volume (calculations made for monochromatic radiation at 280 nm, using literature values for refractive indices, and considering a normal ray incident upon a series of planar refractive index boundaries). However, if the quartz window is wetted by the target water volume, then this loss is reduced to just 4.1%. Thus, it is beneficial to the optical coupling to position and design a UV source such the UV window is wetted by a target water volume.

The interior surface of the irradiation chamber is typically constructed from a material which principally reflects the UV radiation from the UV source and minimally transmits or absorbs the UV radiation.

In another embodiment, the UV source is a LED which is in electrical and thermal connection to a thermal transfer material, such as a metal core printed circuit board (MCPCB), printed circuit board (PCB) or other dielectric material. The thermal transfer material is in direct contact with the fluid in cooling chamber 2, providing a thermal path between the LED and the fluid. In this case, the fluid will provide cooling to the LED if the fluid, e.g., water, temperature is lower than the junction temperature. The thermal transfer material functions as a heat exchange mechanism thermally connected or coupled to the radiation source and to the fluid in the cooling chamber.

In another embodiment, the UV source is a LED which is in electrical and thermal connection to a thermal transfer material, such as a metal core printed circuit board (MCPCB), printed circuit board (PCB) or other dielectric material, which is in contact with a separate, second thermal transfer material in direct contact with the fluid in the irradiation chamber 1, providing a thermal path between the LED and the fluid. In this case, the fluid will provide cooling to the LED if the fluid, e.g., water, temperature is lower than the junction temperature. The second thermal transfer material may be a metal, dielectric, semiconductor, plastic or any other thermally conductive material. The thermal transfer material functions as a heat exchange mechanism thermally connected or coupled to the radiation source and to the fluid in the cooling chamber.

Radiation transmitted through the UV-transparent window to surfaces of the irradiation chamber inhibits biofilm formation on the surfaces and possible microbial contamination in downstream regions of the apparatus. If the apparatus has a fluidic outlet structure optically coupled to the irradiation chamber, either through direct illumination through one or more portholes or other openings in the irradiation chamber or via partial transmission through the material of the chamber, the surfaces of the outlet structure may be irradiated to inhibit biofilm formation thereon. The UV radiation may be used as a biofilm inhibitor within an integrated UV disinfection apparatus, system and method. This may include intelligent control of the apparatus, system, and method with periodic “on cycles” during periods of stagnation, such that a constant bacteriostatic effect may be imparted. On-board sensing of the UV source status could optionally be such as a thermistor, photodiode, or voltage detection scheme. In one embodiment, these sensors could be used to predict the lifetime or operating quality of the UV source. In one embodiment, optical coupling between the irradiation chamber and one or more additional chambers may be accomplished via at least one small porthole through the interior of irradiation chamber to allow for UV radiation to enter the additional chamber(s). The porthole(s) may also be in fluidic connection to the additional chamber(s) and increase fluid communication between the chambers. The radiation transmitted to surfaces of the additional chamber(s) through the porthole(s) and/or via partial transmission through the material of the chamber may inhibit biofilm formation on surfaces of the additional chamber(s) and possible microbial contamination in downstream regions of the apparatus.

In another embodiment, a UV radiation source provides radiation to the interior of the irradiation chamber. The radiation source has a thermal connection to the fluid in the irradiation chamber. This thermal connection is between the backside and/or frontside of at least one heat exchange mechanism thermally connected or coupled to the radiation source and to the fluid in the irradiation chamber. In one embodiment, the heat exchange mechanism is heatsink. A single, quartz optical window is placed over the UV radiation source to protect it from fluid in the irradiation chamber. The UV radiation source is sealed between the heat exchange mechanism and the window such that the window serves to segregate the UV radiation source from the fluid in the irradiation chamber. The irradiation chamber is constructed from a material which principally reflects the UV radiation from the UV source and minimally transmits or absorbs the UV radiation.

In another embodiment, the UV radiation source is thermally connected to a thermal transfer material that is partially or entirely coupled to or mounted inside the interior of the irradiation chamber. The thermal transfer material provides conductive heat transfer from the UV source to the fluid in the irradiation chamber via the interior of the chamber. In one embodiment, the UV source is an LED which is in electrical and thermal connection to the thermal transfer material, such as a metal core printed circuit board (MCPCB), printed circuit board (PCB) or other dielectric material. The thermal transfer material is in direct contact with the fluid in the irradiation chamber providing a thermal path between the LED and the fluid. In this case, the fluid will provide cooling to the LED if the fluid, e.g., water, temperature is lower than the junction temperature. The thermal transfer material functions as a heat exchange mechanism thermally connected or coupled to the radiation source and to the fluid in the cooling chamber.

In another embodiment, the UV source is an LED which is in electrical and thermal connection to the thermal transfer material, such as a metal core printed circuit board (MCPCB), printed circuit board (PCB) or other dielectric material, which is in contact with a separate thermal transfer material in direct contact with the fluid in the irradiation chamber, providing a thermal path between the LED and the fluid. In this case, the fluid will provide cooling to the LED if the fluid, e.g., water, temperature is lower than the junction temperature. The thermal transfer material may be a metal, dielectric, semiconductor, plastic or any other thermally conductive material. The thermal transfer material functions as a heat exchange mechanism thermally connected or coupled to the radiation source and to the fluid in the cooling chamber.

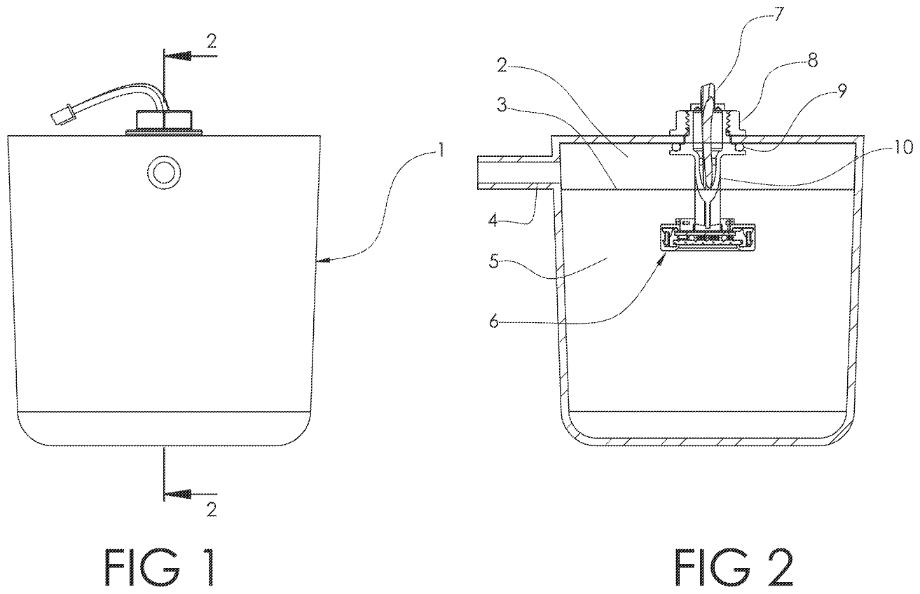

FIGS. 1-4 show one exemplary embodiment of the invention. The irradiation apparatus A includes a three-dimensional irradiation chamber 1 having an inlet port 4 for the flow of a fluid, in this case water 5, containing a material to be irradiated into the chamber. The irradiation chamber may have one or more additional inlet ports for fluid flow into the chamber and/or one or more outlet ports for fluid flow out of the chamber. One or more UV radiation sources inside the irradiation chamber, such as UV radiation sources 17 (FIG. 4), provide radiation to the interior of irradiation chamber. The radiation sources are optically coupled to the water in the irradiation chamber via UV-transparent, quartz optical window 16 (FIG. 4), which is placed over the UV radiation sources to protect them from fluid contacting the window in the irradiation chamber. Gaskets 15 are disposed adjacent to the radiation sources to protect them from the fluid in the irradiation chamber. The gaskets 15 seal the UV radiation sources 17 between the cooling plate heat exchange mechanism 12 and the window 16. End caps 14 securely hold the UV lamp module assembly 6 (FIG. 2) together to prevent fluid from contacting the UV radiation sources.

FIGS. 2-4 show the air 2 and water surface 3 in the irradiation chamber, such that the UV radiation sources and heat exchange mechanism are partially submerged in the water in the irradiation chamber. The heat exchange mechanism is thermally coupled to the radiation sources and to the water in the irradiation chamber. FIG. 2 shows the UV lamp module assembly 6 secured inside the irradiation chamber 1 by retaining nut 8 and sealing O-ring 9 through support stem 10. Power wires 7 inside stem 10 provide electrical current to the UV radiation sources 17.

Heat generated by the UV radiation sources 17 induce convective currents 11 (FIG. 3) within the water in the irradiation chamber. These currents circulate the water from UV occluded regions into those of greater UN exposure, producing a more uniform and effective disinfection effect. The design of the UV lamp module assembly 6 enhances convective cooling and mixing of the water volume. The present invention presents a solution to the challenges of shadowing, packaging the UV source for liquid protection, and UV source cooling.

In another embodiment, the UV source is a micro plasma lamp which is in direct contact with the fluid in the reactor irradiation chamber providing a direct thermal path between the lamp and the fluid. In this case, the fluid will provide cooling to the lamp. A micro plasma lamp UV radiation source provides radiation to the interior of the irradiation chamber. Because the micro plasma lamp is in direct contact with the fluid in the irradiation chamber, it provides a direct thermal path between the lamp and the fluid, thereby cooling the lamp. In one embodiment, the micro plasma lamp is in thermal connection with a thermal transfer material which is in direct contact with the fluid in the irradiation chamber, providing a thermal path between the lamp and the fluid. The thermal transfer material may be a metal, dielectric, semiconductor, plastic or any other thermally conductive material. The thermal transfer material may reflect a portion of the IN radiation from the lamp. In another embodiment, the thermal transfer material is in contact with a separate thermal transfer material which is in direct contact with the fluid in the irradiation chamber, providing a thermal path between the lamp and the fluid. In these cases, the fluid will provide cooling to the lamp. As such, the embodiment may be used as an irradiation chamber in the other irradiation apparatus shown and described herein.

In another embodiment, the invention provides a plurality of UV radiation sources and a plurality of irradiation chambers, each with at least one inlet and one outlet port. Each UV radiation source is primarily optically coupled to a single irradiation chamber. All irradiation chambers are fluidically coupled such that all fluid that passes through any irradiation chamber also passes through the other irradiation chambers. In this way, the fluidic flux through the irradiation chambers is equal to the sum of fluidic fluxes through all the irradiation chambers. In addition, all UV sources are thermally coupled to the fluidic flux via the interior of the irradiation chambers.

In another embodiment, the invention provides a plurality of UV radiation sources and a plurality of irradiation chambers, each with at least one inlet and one outlet port. Each UV radiation source is primarily optically coupled to a single irradiation chamber. All of the UV radiation sources are thermally coupled to all the irradiation chamber. One or more of the irradiation chambers is in fluidic connection, where the outlet of one chamber is the inlet for another chamber.

In the embodiments described above, the plurality of irradiation chambers may be fluidically coupled such that all fluid that passes through any irradiation chamber also passes through the other irradiation chambers. Just as multiple irradiation chambers may be fluidically coupled, forming a single unit, sets of these individual units may be arrayed in parallel or series combinations where the inlet to each unit is composed of a fraction of the total inlet flow (parallel case) or the entire flow (series case), or a blend of series and parallel configurations of each unit.

In another embodiment, the transfer of heat from the UV source to the fluidic flux is accomplished via conductive heat transfer through a nominally flat surface that is incorporated into the surface of a chamber, in thermal contact with the fluidic flux within that chamber. For example, in the embodiments shown in FIGS. 1-4, the transfer of heat from the UV source to the fluid in the irradiation chamber is accomplished via conductive heat transfer through a nominally flat surface of the heatsink incorporated into the outer surface of the irradiation chamber and the inner surface of the chamber, which is in thermal contact with the fluidic flux within the chamber.

In another embodiment, the transfer of heat from the UV source to the fluidic flux is accomplished via conductive heat transfer through a porous structure placed in the flow path of some or all of the fluidic flux. The porous structure may be designed such that the surface area is maximized to provide for efficient conductive heat transfer to the fluidic flux. The porous structure used for maximizing conductive heat transfer may also promote turbulent mixing of the fluidic flux and/or laminar flow characteristics in the fluidic flux.

In yet another embodiment, two three-dimensional chambers have at least one inlet and at least one outlet port for the flow of a fluid into and out of the chamber. The UV source is a planar source such as a micro plasma lamp, emitting UV radiation from both sides. The UV source is situated between the irradiation chambers and provides radiation to both chambers. In one embodiment, the two chambers are in fluidic connection, where the inlet of one of the chambers is the outlet for the other chamber. In another embodiment, each side of the planar UV source serves as a portion of the sidewall of each chamber.

In another embodiment, the irradiation apparatus includes two three-dimensional irradiation chambers, each having an inlet port and an outlet port for the flow of a fluid into and out of the chambers. The irradiation chambers are in fluidic connection and in fluid communication, with a port functioning as the outlet port for one chamber and the inlet port for the other irradiation chamber. The UV radiation source is a micro plasma lamp that provides radiation to the interior of both irradiation chambers. The UV source is situated between the irradiation chambers and provides radiation to both chambers. Each side of the planar UV source serves as a portion of the sidewall of each chamber. The UV radiation source has a quartz sleeve or optical window covering each of its sides to protect it from fluid in the irradiation chambers. The UV radiation source is sealed between the windows such that the windows serve as a portion of the pressure vessel for the disinfection system and to segregate the UV radiation source from the fluid in the irradiation chambers.

In another embodiment of the invention, the UV source described herein may comprise a UV emitter embedded inside an environmentally sealed housing which partially or completely encloses the UV emitter between a thermal transfer material or conductor such as a metal core printed circuit board, and a UV transparent window. In another embodiment, the sealed housing comprises a principally UV transparent window and a heatsink, such as a principally thermally conducting cup, that combine to form an enclosed volume in which one or more UV LEDs on a circuit board is located and which is in thermal connection to the cup. A potting compound fills the void between the thermally conductive cup and the window, less a small keep out area around the perimeter of the LEDs. In one embodiment, the thermally conductive cup is created by deformation of a single metal sheet. The thermally conductive cup may have one or more ports for electrical connection entry and/or exit and/or for the injection of a liquid potting compound. In another embodiment, the thermally conductive cup comprises at least one face intended principally for thermal transfer to/from the UV emitter.

In other embodiments of the invention, the optically transparent window is made of quartz or sapphire or a principally UV transparent polymer. The potting compound may principally retain the optically transparent window in the thermally conductive cup and serve as a structural component to the assembly. The UV emitter may comprise a UV radiation source mounted on a substrate with a control system further mounted on the substrate. The UV radiation source may comprise at least one of an LED, a plasma discharge source, or a solid-state phosphor emission device, or combinations thereof. The substrate may comprise a printed circuit board. The substrate may be designed to create an efficient thermal path between the UV radiation source and an external thermal reservoir. The substrate may provide a means of preventing contact between the potting compound and UV radiation source. The substrate may provide a means to fix relative positioning of the UV radiation source and the optically transparent window. A control system may comprise a constant-current source or a constant-current sink.

The present invention has numerous potential applications. Primarily, this may be considered as a means for treating or maintaining microbial quality of potable water tanks; however, the breadth of application is far more substantial. The storage of water and other fluids is required for numerous processes, including but not limited to, crop irrigation, coolant loops & injection systems, greywater, cleaning fluids, humidifiers, dehumidifiers, flushing & quenching systems, wastewater treatment, food processing and dispensing, pharmaceutical production, etc. In such applications, the objective may be to control microbial contamination for the purposes of avoiding disease, or for the avoidance of other unfavorable effects of bacterial or mold growth such as aesthetics, clogging, corrosion, rotting, digestion, etc. Further, the application of an in-tank disinfection system may be desired during nominal operation, e.g. when the tank is full of a target fluid, or as a means of maintaining operational readiness during dormancy, when the tank surfaces themselves may become the primary disinfection target.

Although the invention is illustrated and described herein with reference to certain embodiments and examples thereof, it will be readily apparent to those skilled in the art that other embodiments and examples may perform similar functions and/or achieve like results. Likewise, it will be apparent that other applications of the disclosed technology are possible. All such equivalent embodiments, examples, and applications are within the spirit and scope of the invention and are intended to be covered by the following claims.

We claim:

1. An irradiation apparatus comprising:
at least one irradiation chamber for a fluid containing a material to be irradiated, said chamber having at least one inlet port for fluid flow into the chamber;
one or more UV radiation sources inside the at least one irradiation chamber optically coupled to the fluid in the at least one irradiation chamber via at least one UV-transparent window in contact with the fluid in the irradiation chamber;
one or more seals or gaskets disposed adjacent to the one or more radiation sources to protect the one or more radiation sources from the fluid in the irradiation chamber; and
at least one heat exchange mechanism inside the at least one irradiation chamber thermally coupled to the one or more radiation sources and to the fluid in the at least one irradiation chamber;
wherein the one or more UV radiation sources and the at least one heat exchange mechanism are at least partially submerged in the fluid in the irradiation chamber.

2. The irradiation apparatus of claim 1, wherein the heat exchange mechanism comprises one or more of a printed circuit board, a metal core printed circuit board, a thermoelectric cooling device, a vapor chamber, a heatsink, a heat dissipation structure, a thermal transfer material, a material thermally coupled to a fluid.

3. The irradiation apparatus of claim 2, wherein the heat exchange mechanism is a heatsink or a thermal transfer material, or combinations thereof.

4. The irradiation apparatus of claim 1, further comprising one or more sensors which is used to dynamically control the power to the one or more UV radiation sources based on one or more sensor readings.

5. The irradiation apparatus of claim 1 further comprising circuitry which monitors the status of the one or more UV radiation sources and provides feedback to monitoring circuitry.

6. The irradiation apparatus of claim 1, wherein the one or more UV radiation sources comprise one or more UV-C radiation sources, or a combination thereof.

7. The irradiation apparatus of claim 1, wherein the one or more UV radiation sources comprise a plurality of radiation sources arranged in an array.

8. The irradiation apparatus of claim 1, wherein one or more wavelengths of the one or more UV radiation sources are dynamically adjustable.

9. The irradiation apparatus of claim 1, wherein one or more wavelengths of the one or more UV radiation sources are selected based on an identification of a contaminant in the material to be irradiated.

10. The irradiation apparatus of claim 1, wherein the one or more UV radiation sources deliver one or more wavelengths to the material to be irradiated that induce fluorescence in the material to be irradiated thereby allowing for the identification of the contaminant in the material to be irradiated.

11. The irradiation apparatus of claim 1, wherein the one or more UV radiation sources deliver a combination of wavelengths to the material to be irradiated.

12. The irradiation apparatus of claim 1, wherein the one or more UV radiation sources comprise a micro plasma lamp.

13. The irradiation apparatus of claim 1, comprising a plurality of UV radiation sources and a plurality of irradiation chambers, each with at least one inlet and one outlet port, and all of the UV radiation sources are thermally coupled to the irradiation chambers.

14. The irradiation apparatus of claim 1, wherein a portion of the radiation from the one or more radiation sources is transmitted to surfaces of one or more irradiation chambers to inhibit biofilm formation on the surfaces.

15. A method for irradiating a fluid containing a material to be irradiated disposed in an irradiation chamber, the irradiation method comprising:
(1) providing an irradiation apparatus comprising:
at least one irradiation chamber for a fluid containing a material to be irradiated, said chamber having at least one inlet port for fluid flow into the chamber;
one or more UV radiation sources inside the at least one irradiation chamber optically coupled to the fluid in the at least one irradiation chamber via at least one UV-transparent window in contact with the fluid in the irradiation chamber;
one or more seals or gaskets disposed adjacent to the one or more radiation sources to protect the one or more radiation sources from the fluid in the irradiation chamber; and
at least one heat exchange mechanism inside the at least one irradiation chamber thermally coupled to the one or more radiation sources and to the fluid in the at least one irradiation chamber;
wherein the one or more UV radiation sources and the at least one heat exchange mechanism are at least partially submerged in the fluid in the at least one irradiation chamber; and
(2) irradiating a fluid containing a material to be irradiated using said irradiating apparatus.

16. The irradiation method of claim 15, wherein the heat exchange mechanism comprises one or more of a printed circuit board, a metal core printed circuit board, a thermoelectric cooling device, a vapor chamber, a heatsink, a heat dissipation structure, a thermal transfer material, a material thermally coupled to a fluid.

17. The irradiation method of claim 15, wherein the heat exchange mechanism is coated with a water, medical or food safe material.

18. The irradiation method of claim 15, further comprising one or more sensors which is used to dynamically control the power to the one or more UV radiation sources based on one or more sensor readings.

19. The irradiation method of claim 15, further comprising circuitry which monitors the status of the one or more UV radiation sources and provides feedback to monitoring circuitry

20. The irradiation method of claim 15, wherein the one or more UV radiation sources comprise one or more UV-C radiation sources, or a combination thereof.

21. The irradiation method of claim 15, wherein the one or more UV radiation sources comprise a plurality of radiation sources arranged in an array.

22. The irradiation method of claim 15, wherein one or more wavelengths of the one or more UV radiation sources are dynamically adjustable.

23. The irradiation method of claim 15, wherein one or more wavelengths of the one or more UV radiation sources are selected based on an identification of a contaminant in the material to be irradiated.

24. The irradiation method of claim 15, wherein the one or more UV radiation sources deliver one or more wavelengths to the material to be irradiated that induce fluorescence in the material to be irradiated thereby allowing for the identification of the contaminant in the material to be irradiated.

25. The irradiation method of claim 15, wherein the one or more UV radiation sources deliver a combination of wavelengths to the material to be irradiated.

26. The irradiation method of claim 15, wherein the one or more UV radiation sources comprise a micro plasma lamp.

27. The irradiation method of claim 15, comprising a plurality of UV radiation sources and a plurality of irradiation chambers, each with at least one inlet and one outlet port, and all of the UV radiation sources are thermally coupled to the irradiation chambers.

28. The irradiation method of claim 15, wherein a portion of the radiation from the one or more radiation sources is transmitted to surfaces of one or more irradiation chambers to inhibit biofilm formation on the surfaces.

29. An irradiation apparatus comprising:
at least one irradiation chamber for a fluid containing a material to be irradiated, said chamber having at least one inlet port for fluid flow into the chamber;
one or more UV radiation sources inside the at least one irradiation chamber optically coupled to the fluid in the at least one irradiation chamber via at least one UV-transparent window in contact with the fluid in the irradiation chamber;
one or more seals or gaskets disposed adjacent to the one or more radiation sources to protect the one or more radiation sources from the fluid in the irradiation chamber; and
at least one heat exchange mechanism inside the at least one irradiation chamber thermally coupled to the one or more radiation sources and to the fluid in the at least one irradiation chamber;
wherein the one or more UV radiation sources and the at least one heat exchange mechanism are at least partially submerged in the fluid in the irradiation chamber, and the UV lamp module assembly is constructed in such a way as to preferentially create convection currents in order to mix a stagnant tank volume.
